(19) United States
(12) Patent Application Publication
(10) Pub. No.: US 2003/0217386 A1
(43) Pub. Date: Nov. 20, 2003

(54) Transgenic fodder plants with an increased leaf starch content

(75) Inventors: Claus Frohberg, Berlin (DE); Martin Leube, Potsdam (DE); Isabel Rumpler, Potsdam (DE)

Correspondence Address: BIRCH STEWART KOLASCH & BIRCH, PO BOX 747, FALLS CHURCH, VA, 22040-0747, US

(73) Assignee: PlantTec Biotechnologie GmbH Forschung & Entwicklung, Potsdam (DE)

(21) Appl. No.: 10/371,077

(22) Filed: Feb. 21, 2003

(30) Foreign Application Priority Data
Feb. 26, 2002 (DE) 10208133.6

(51) Int. Cl. A01H 1/00; C12N 15/82

(52) U.S. Cl. 800/284

(57) ABSTRACT

There is described a method for generating transgenic fodder plants which are genetically modified, where the genetic modification leads to a reduced activity of an R1 protein in comparison to corresponding wild-type plants which have not been genetically modified. Such transgenic fodder plants are distinguished by their substantially increased leaf starch content in comparison with wild-type plants.

The present invention relates to a method for generating transgenic fodder plant cells or fodder plants which are genetically modified, the genetic modification in the cells or plants bringing about the reduced activity of an R1 protein in comparison to corresponding wild-type plant cells or plants which have not been genetically modified. Plants generated by this method synthesize a modified leaf starch, the starch content in the leaves of the plants being up to 1 000% higher than that of wild-type plants which have not been genetically modified. This starch is furthermore preferably characterized in that it has a reduced phosphate content. Furthermore, the present invention relates to transgenic fodder plant cells and fodder plants with a reduced activity of an R1 protein in comparison with wild-type plants or plant cells.

Fodder plants are mainly understood as meaning the clover-like fodder plants (=fodder legumes) and the fodder grasses. Both differ from most important crop plants by the fact that the crop product of interest is not predominantly reproductive organs such as seeds, fruits or tubers, but the entire aerial plant biomass. The fodder legumes include the clover species (such as, for example, the forms of Trifolium, Medicago and Lotus), the vetch species (such as Vicia and Coronilla), furthermore sainfoin (Esparsette) and serradella (Ornithopus). The fodder grasses include the various types of darnel, meadow-grass, cattail, Avena, cocksfoot grass and bentgrass, and the fescue species, inter alia.

The demands made of feed plants are an increased feed value. In addition to the consumption capacity, this complex term also encompasses the aspect of digestibility and the content of valuable constituents.

Only animals are capable of converting fodder plants properly. The farmer expects that an improved feed quality of the green fodder, which is provided in fresh form or as silage, will lead to better conversion of the material fed and that the energy requirements in livestock feeding will be met more specifically. However, the differences between ruminants and nonruminants must be taken into consideration. In cattle, the supplementation with concentrates only meets part of the energy requirements since the specific digestion processes in ruminants require a minimum of structured roughage. Fodder plants should therefore have a high utilizable energy content and ensure very good consumption. High energy requirement is in proportion to digestibility. Consumption depends on the rate of passage through the gut. Fodder plants, especially fodder legumes such as Trifolium repens and Lolium perenne, are very high in protein, but only contain a small amount of nonsoluble carbohydrates (NSC). As a consequence, most of the ammonium produced cannot be metabolized, but, in order to avoid poisoning, is transported to the liver, converted into uric acid and then excreted. This leads to nitrogen loss and simultaneously large amounts of excreted ammonia.

Livestock producers expect that an increased leaf starch content will lead to an improved carbon/nitrogen ratio (C/N) and, as a consequence, better digestibility and energy uptake and, eventually, higher production of meat/milk in combination with less excreted ammonia.

Ritte et al. (Plant J. 21 (4), (2000), 387-391) were able to demonstrate that, in potato plants, the Solanum tuberosum R1 protein binds reversibly to starch granules, the strength of binding to the starch granules depending on the metabolic status of the plant. In potato plants, the protein in its starch-granule-bound form occurs predominantly in leaves kept in the dark. After exposure of the leaves to light, in contrast, the protein occurs predominantly in the soluble form and is not bound to the starch granule.

Studies on transgenic potatoes have revealed that a reduced expression of the R1 gene leads to an increased starch content in the leaves of potato plants. This entails an increase in dry matter by 50-90%. Similar results were obtained in studies on leaves of transgenic tobacco plants in which the R1 gene was suppressed by antisense suppression (Lorberth et al., Nature Biotechnology 16 (1998), 473-477). However, no increased starch content in fodder plants have been shown as yet; in addition, the relevant gene sequences were not available.

The present invention is based on the object of providing a method by means of which transgenic fodder plants which are adapted better to the requirements of agriculture can be generated, in particular in as far as they have a higher utilizable energy content.

This object is achieved by providing the use forms specified in the patent claims.

The present invention thus relates to a method for generating a transgenic fodder plant with an increased leaf starch content in comparison to corresponding wild-type plants, where

(a) a cell of a fodder plant is genetically modified by introducing a foreign nucleic acid molecule whose presence or expression leads to a reduced activity of an R1 protein which occurs endogenously in the plant;

(b) a plant is regenerated from the cell generated in step (a); and

(c) if appropriate, further plants are generated starting from the plant generated in step (b).

In the present context, the term “transgenic” means that the plants generated by the method according to the invention differ, owing to a genetic modification, in particular the introduction of a foreign nucleic acid molecule, with regard to their genetic information from corresponding plant cells which have not been genetically modified and can be distinguished from them. In particular, the term means that the cells of these plants contain a foreign nucleic acid molecule which does not occur naturally in corresponding wild-type plants which have not been genetically modified or which occurs at a locus in the genome of the cells at which it does not occur naturally. In this context, the term “wild-type plants” means a plant of the same species which, however, contains no corresponding genetic modification, in particular no genetic modification in connection with the R1 gene. In this context, “foreign” nucleic acid molecule means that the nucleic acid molecule is heterologous with regard to the species to which the plants into whose cells the nucleic acid molecule is introduced belong, or that, if the nucleic acid molecule is homologous to this plant species, it occurs in a genetic context in which it does not occur naturally in the plant cells. This means that it occurs at a different locus in the genome of the plant cell and/or is linked to sequences to which it is not linked naturally in the plant cells. Whether a plant or plant cell is transgenic can be verified by methods with which the skilled worker is familiar, for example Southern blot analysis.

For the purposes of the present invention, the term “fodder plants” refers to plants which are grown for the purpose of being utilized as animal feed.

The “fodder plants” may take the form of monocotyledonous or else dicotyledonous fodder plants. In particular, “fodder plants” is understood as meaning what are known as fodder legumes. These are plants which belong to the superorder of the Fabanae, in particular the Fabales (=Leguminosae). Preferred in this context are plants of the Fabaceae family. Especially preferred are clover species, for example plants of the genus Trifolium, such as, for example, T. repens, T. pratense, T. hybridum or T. incarnatum. Others which are especially preferred are Lucerne (Medicago sativa), Lotus types, for example Lotus corniculatus; sainfoin (Onobrychis viciifolia), Serradella (Ornithopus sativus), lupin, for example Lupinus angustifolius or Lupinus luteus, and vetch species, such as, for example, Vicia species (for example Vicia faba) or Coronilla species.

Fodder plants are furthermore understood as meaning fodder grasses. These are plants of the order Poales, in particular those of the Poaceae family. Preferred in this context are plants of the genus Lolium (for example Lolium perenne), Poa (for example P. pratensis, P. palustris, P. longifolia), Phleum (for example P. nodusum, P. pratense), Dactylis (for example D. glomerata), Agrostis (for example A. tennuis, A. stolonifera), Festuca (for example F. pratensis, F. rubra), Bromus (for example Bromus molli) and Avena species.

The term “increased leaf starch content” refers to the fact that the amount of starch formed in the leaves markedly exceeds the amount formed in the leaves of corresponding wild-type plants. The leaf starch content of the fodder plants generated by the method according to the invention is increased by at least 10-50%, preferably by at least 50-100%, in particular by 100-500% and very particularly preferably by at least 500-1 000% in comparison with the leaf starch content of corresponding wild-type plants.

The starch content in the leaf is preferably measured in nmol/g dry matter. Methods for determining the leaf starch content are known to the skilled worker and described for example in the examples which follow.

For the purposes of the present invention, the term “genetically modified” refers to the fact that the genetic information of the plant cell is modified in comparison with corresponding cells of a wild-type plant by the introduction of a foreign nucleic acid molecule and that the presence and/or the expression of the foreign nucleic acid molecule results in an altered phenotype of the plant regenerated from this plant cell. In this context, altered phenotype preferably means a measurable difference of one or more functions of the cell and/or the plant. By way of example, in genetically modified plant cells according to the invention, the activity of an R1 protein which occurs endogenously in the plant cell is reduced in comparison with corresponding plant cells of wild-type plants which have not been genetically modified.

For the purposes of the present invention, the term “reduced activity” refers to a reduced expression of endogenous genes which encode R1 proteins, and/or to a reduced amount of R1 protein in the cells and/or a reduced biological activity of the R1 proteins in the cells.

In this context, “reduced expression” refers to the fact that plants generated by the method according to the invention, or the cells of these plants, contain fewer transcripts which encode an R1 protein than corresponding wild-type plant cells. “Reduced expression” can be determined for example by measuring the amount of transcripts encoding R1 proteins, for example by Northern blot analysis or RT-PCR. Reduction in this context preferably means a reduced amount of transcripts by at least 50%, in particular by at least 70%, preferably by at least 85% and especially preferably by at least 95% in comparison with corresponding cells which have not been genetically modified. In a very especially preferred embodiment, the reduction amounts to 100%, i.e. the expression of R1 genes in the plants (plant cells) is completely repressed, and no R1 protein whatsoever is synthesized in the cells.

“Reduced amount” of R1 protein means that the content of R1 protein in the plants or in the cells of the plants which are generated by the method according to the invention is less than in corresponding wild-type plants or wild-type plant cells. Methods for determining the R1 protein content are known to the skilled worker.

Thus, the reduced amount of R1 proteins can be determined for example by Western blot analysis. In this context, reduction means a reduced amount of R1 protein by at least 50%, in particular by at least 70%, preferably by at least 85%, especially preferably by at least 95% and very especially preferably by 100% in comparison to corresponding wild-type plants or cells which have not been genetically modified.

For the purposes of the present invention, the term “R1 gene” is understood as meaning a nucleic acid sequence (for example RNA or DNA, such as cDNA or genomic DNA) which encodes a “R1 protein”. For the purposes of the present invention, the term “R1 protein” is understood as meaning proteins which have been described for example in Lorberth et al. (Nature Biotech. 16 (1998), 473-477) and in the international patent applications WO 98/27212, WO 00/77229 and WO 00/28052 and which have specific traits. Important traits of R1 proteins are

(i) their localization in the plastids (such as chloroplasts, amyloplasts) of plant cells;

(ii) their property of occurring, in the plastids, partly in free form and partly bound to starch granules;

(iii) their ability of influencing the degree of starch phosphorylation in plants, inasmuch as an increased activity of the R1 protein in plants leads to an increased phosphate content of the starch synthesized in the plants, and a reduced activity of the R1 protein in plants leads to a reduced phosphate content of the starch synthesized in the plants. In this context, the phosphate content relates to the C-6-phosphate content. It is preferably indicated as nmol/mg dry leaf starch. It can be determined as described in the examples section which follows; and

(iv) their ability of, when expressed in E. coli cells, leading to phosphorylation of the bacterial glycogen. This ability can be assayed for example as described in WO 98/27212.

For the purposes of the present invention, an R1 protein is preferably understood as meaning a protein which has the abovementioned characteristics and which is encoded by a nucleic acid molecule encompassing a nucleotide sequence which hybridizes with the coding region of SEQ ID NO: 7 or its complementary strand. In this context, the term “hybridization” refers to hybridization under conventional hybridization conditions, preferably under stringent conditions as are described, for example, in Sambrook et al. (1989, Molecular Cloning, A Laboratory Manual, 2nd ed. Cold Spring Harbor Laboratory Press, Cold Spring Harbor, N.Y.). Such hybridizing nucleic acid molecules can be isolated for example from genomic libraries or from cDNA libraries of plants.

Such nucleic acid molecules can be identified and isolated using the nucleic acid molecules shown in SEQ ID NO: 7 or parts of these molecules or the reverse complements of these molecules, for example by means of hybridization by standard methods (see, for example, Sambrook and Russel, Molecular Cloning, A Laboratory Manual, 3rd ed. Cold Spring Harbor Laboratory Press (2001), Cold Spring Harbor, N.Y.).

Examples of nucleic acid molecules which can be used as hybridization probe are those with exactly or essentially the sequence shown under SEQ ID NO: 7 or part of this sequence. The DNA fragments used as hybridization probe may also take the form of synthetic DNA fragments which have been generated with the aid of the customary DNA synthesis techniques and whose sequence agrees essentially with the nucleic acid sequence shown in SEQ ID NO: 7.

“Hybridization” preferably means that at least 60%, preferably at least 80%, especially preferably at least 90% and very especially preferably at least 95% homology, i.e. sequence identity, exists between the molecules in question.

The degree of homology is determined by comparing the nucleotide sequence in question with the coding region shown in SEQ ID NO: 7. If the sequences to be compared differ in length, the degree of homology preferably relates to the percentage of nucleotides in the shorter sequence which are identical to nucleotides in the longer sequence. The homology can be determined by customary methods, in particular using computer programs such as, for example, the DNASTAR program in combination with Clustal W analysis. This program is obtainable from DNASTAR, Inc. 1228 South Park Street, Madison, Wis. 53715 or from DNASTAR, Ltd., Abacus House, West Ealing, London W13 OAS UK (support @dnastar.com), and is accessible via the EMBL server.

If the analytical method Clustal is used for determining homology, in particular for determining whether a sequence is identical, for example 80% identical, to a reference sequence, the settings are preferably as follows: Matrix: blosum 30; Open gap penalty: 10.0; Extend gap penalty: 5.0; Delay divergent: 40; Gap separation distance 8.

Generation of the plant cells with reduced R1 activity in accordance with the method according to the invention can be achieved by various methods with which the skilled worker is familiar, for example by those which lead to inhibition of the expression of endogenous genes which encode an R1 protein. These include, for example, the expression of a corresponding antisense RNA, the provision of molecules or vectors which confer a cosuppression effect, the expression of a suitably constructed ribozyme which specifically cleaves transcripts encoding an R1 protein, or what is known as “in-vivo mutagenesis”.

Furthermore, reduced R1 activity in the plant cells can also be brought about by the simultaneous expression of sense and antisense RNA molecules of the target gene in question which is to be repressed, preferably of the R1 gene. Moreover, it is known that the formation, in planta, of RNA duplex molecules of promoter sequences in trans can lead to methylation and transcriptional inactivation of homologous copies of this promoter (Mette et al., EMBO J. 19 (2000), 5194-5201).

Furthermore, the use of introns, i.e. of noncoding regions of genes which encode R1 proteins, is also feasible for achieving an antisense or a cosuppression effect. The use of intron sequences for inhibiting the gene expression of genes which encode proteins of starch biosynthesis has been described for example in international patent applications WO 97/04112, WO 97/04113, WO 98/37213 and WO 98/37214.

In a preferred embodiment of the method according to the invention, the foreign nucleic acid molecule is selected from the group consisting of

(a) DNA molecules which encode at least one antisense RNA which brings about reduced expression of endogenous genes which encode R1 proteins;

(b) DNA molecules which, via a cosuppression effect, lead to reduced expression of endogenous genes which encode R1 proteins;

(c) DNA molecules which encode at least one ribozyme which specifically cleaves transcripts of endogenous genes which encode R1 proteins;

(d) nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of R1 proteins; and

(e) DNA molecules which simultaneously encode at least one antisense RNA and at least one sense RNA, where the antisense RNA and the sense RNA form an RNA duplex which brings about a reduced expression of endogenous genes which encode R1 proteins.

The skilled worker knows how to achieve an antisense effect or a cosuppression effect. The method of antisense inhibition is described, for example, in Krol et al. (Nature 333 (1988), 866-869), Krol et al. (Gene 72 (1988), 45-50), Mol et al. (FEBS Letters 268 (2), (1990), 427-430), Smith et al. (Plant Mol. Biol. 14 (1990), 369-379) and Sheehy et al. (Proc. Natl. Acad. Sci. USA 85 (1988), 8805-8809). The method of cosuppression inhibition has been described, for example, in Jorgensen (Trends Biotechnol. 8 (1990), 340-344), de Carvalho Niebel et al. (Curr. Top. Microbiol. Immunol. 197 (1995), 91-103), Flavell et al. (Curr. Top. Microbiol. Immunol. 197 (1995), 43-56), Palauqui and Vaucheret (Plant. Mol. Biol. 29 (1995), 149-159), Vaucheret et al. (Mol. Gen. Genet. 248 (1995), 311-317) and in de Borne et al. (Mol. Gen. Genet. 243 (1994), 613-621).

The expression of ribozymes for reducing the activity of certain enzymes in cells is likewise known to the skilled worker and described, for example, in EP-B1 0321 201. The expression of ribozymes in plant cells has been described, for example, in Feyter et al. (Mol. Gen. Genet. 250 (1996), 329-338).

Furthermore, the reduced R1 activity in the plant cells can also be achieved, as mentioned above, by what is known as “in-vivo mutagenesis”, where a chimeric RNA-DNA oligonucleotide (“chimeroplast”) is introduced into cells by cell transformation (Beetham, Kipp et al., Poster Session at the “5^{th} International Congress of Plant Molecular Biology”, Sep. 21-27, 1997, Singapore; Dixon and Arntzen, Meeting report on “Metabolic Engineering in Transgenic Plants”, Keystone Symposia, Copper Mountain, Colo., USA, TIBTECH 15 (1997), 441-447; international patent application WO 95/15972; Kren et al., Hepatology 25 (1997), 1462-1468; Cole-Strauss et al., Science 273 (1996), 1386-1389).

Part of the DNA component of the RNA-DNA oligonucleotide is homologous to a nucleic acid sequence of an endogenous R1 gene, but contains a mutation in comparison with the nucleic acid sequence of an endogenous R1 gene or contains a heterologous region which is flanked by the homologous region. Owing to this pairing of the homologous regions of the RNA-DNA oligonucleotide and of the endogenous nucleic acid molecule, followed by homologous recombination, the heterologous region or the mutation obtained in the DNA component of the RNA-DNA oligonucleotide can be transferred into the genome of a plant cell. The mutation is chosen in such a way that it, upon expression of the corresponding sequence, leads to a reduced activity of an R1 protein, i.e. preferably to a reduced expression of the gene, to a reduced synthesis of an R1 protein or to a reduced biological activity of an R1 protein, for example owing to the expression of inactive proteins, in particular of dominant-negative mutants.

Furthermore, the reduced R1 activity in the plant cells may also be brought about by the simultaneous expression of sense and antisense RNA molecules of the target gene to be repressed, i.e. of the R1 gene. This can be achieved for example by using chimeric constructs which contain inverted repeats of the target gene to be repressed, or of parts of the target gene. In this context, the chimeric constructs encode sense and antisense RNA molecules of the target gene. Sense and antisense RNA are synthesized simultaneously in planta as an RNA molecule, it being possible for sense and antisense RNA to be separated from each other by a spacer, forming an RNA duplex. It has been demonstrated that the introduction of inverted-repeat DNA constructs into the genome of plants is a highly effective method of repressing the genes which correspond with the inverted-repeat DNA constructs (Waterhouse et al., Proc. Natl. Acad. Sci. USA 95 (1998), 13959-13964; Wang and Waterhouse, Plant Mol. Biol. 43 (2000), 67-82; Singh et al., Biochemical Society Transactions 28, part 6 (2000), 925-927; Liu et al., Biochemical Society Transactions 28, part 6 (2000), 927-929); Smith et al., Nature 407 (2000), 319-320; international patent application WO 99/53050). Sense and antisense sequences of the target gene(s) may also be expressed separately of one another by means of identical or different promoters (Nap et al., 6^{th} International Congress of Plant Molecular Biology, Quebec, Jun. 18-24, 2000; Poster S7-27, paper Session S7).

Thus, reduced R1 activity in the plant cells can also be achieved by generating RNA duplexes of R1 genes. For this purpose, it is preferred to introduce, into the genome of plants, inverted repeats of DNA molecules of R1 genes or R1 cDNAs, the DNA molecules to be transcribed (R1 gene or R1 cDNA or fragments thereof) being under the control of a promoter which governs the expression of said DNA molecules.

Moreover, it is known that the formation of RNA duplexes of promoter DNA molecules in plants in trans can lead to methylation and a transcriptional inactivation of homologous copies of these promoters, which are hereinbelow referred to as target promoters (Mette et al., EMBO J. 19 (2000), 5194-5201). Thus, it is possible, via the inactivation of the target promoter, to reduce the gene expression of a particular target gene (for example of the R1 gene) which is naturally under the control of this target promoter. This means that the DNA molecules which comprise the target promoters of the genes to be repressed (target genes) are now not used as control elements for expressing genes or cDNAs, but as transcribable DNA molecules themselves, which is in contrast to the original function of promoters in plants.

Constructs which are preferably used for generating the target promoter RNA duplexes in planta, where they may exist in the form of RNA hairpin molecules, are those comprising inverted repeats of the target promoter DNA molecules, the target promoter DNA molecules being under the control of a promoter which governs the gene expression of said target promoter DNA molecules. These constructs are subsequently introduced into the genome of plants. In planta, the expression of the inverted repeats of said target promoter DNA molecules leads to the formation of target promoter RNA duplexes (Mette et al., EMBO J. 19 (2000), 5194-5201), by which the target promoter can be inactivated.

The promoter regions of the R1 genes from the plant species in question can be isolated and characterized by screening suitable genomic DNA libraries. Known cDNA or genomic fragments of the R1 genes can be used as homologous probes in this context. The generation and screening of genomic DNA libraries is known to the skilled worker and described in Sambrook and Russell (Molecular Cloning, 3rd edition (2001), Cold Spring Harbour Laboratory Press, NY).

Furthermore, the skilled worker knows that a reduced activity of one or more R1 proteins can be achieved by the expression of nonfunctional derivatives, in particular trans-dominant mutants of such proteins, and/or by the expression of antagonists/inhibitors of those proteins. Antagonists/inhibitors of those proteins encompass for example antibodies, antibody fragments or molecules with similar binding properties. For example, a cytoplasmic scFv antibody was employed for modulating the activity of the phytochrome A protein in genetically modified tobacco plants (Owen, Bio/Technology 10 (1992), 790-794; Review: Franken et al., Current Opinion in Biotechnology 8 (1997), 411-416; Whitelam, Trends in Plant Science 1 (8), (1996), 268-272).

In an especially preferred embodiment of the method according to the invention, the gene which occurs endogenously in the fodder plant and which encodes an R1 protein is selected from the groups consisting of:

(a1) nucleic acid molecules which encompass the coding region of the nucleotide sequence shown under SEQ ID NO: 1 or the coding region of the insertion in plasmid DSM 14707;

(b1) nucleic acid molecules which encode a protein encompassing the amino acid sequence shown in SEQ ID NO: 2 or the amino acid sequence encoded by the insertion of plasmid DSM 14707;

(c1) nucleic acid molecules whose sequence has at least 90% homology with the nucleic acid molecules mentioned under (a1) or (b1);

(d1) nucleic acid molecules whose sequence is degenerate in comparison with the sequences of the nucleic acid molecules mentioned under (a1), (b1) or (c1) owing to the genetic code; and

(e1) parts or fragments of the nucleic acid molecules mentioned under a1) to d1) selected from the group consisting of fragments with a length of at least 25, 50, 100 bp, preferably with a length of at least 250 bp, especially preferably with a length of at least 500 bp.

In one such embodiment, the plant generated in the method according to the invention is preferably a plant of the genus Trifolium, especially preferably of the species Trifolium repens. Furthermore, the foreign nucleic acid molecule preferably employed in such an embodiment is a nucleic acid molecule which is a nucleic acid molecule as defined above under (a1) to (d1), part of such a molecule which is long enough in order to achieve the desired effect, or a nucleic acid molecule which is complementary to such a nucleic acid molecule or part thereof.

The sequence shown under SEQ ID NO: 1 originates from Trifolium repens and constitutes a partial cDNA sequence which encodes an R1 protein. The coding region shown has 75% homology with the coding region shown in SEQ ID NO: 7, which encodes the potato R1 protein.

In another preferred embodiment, the gene which occurs endogenously in the fodder plant and which encodes an R1 protein is a gene selected from the group consisting of:

(a2) nucleic acid molecules which encompass the coding region of the nucleotide sequence shown under SEQ ID NO: 3 or the coding region of the insertion in plasmid DSM 14633;

(b2) nucleic acid molecules which encode a protein encompassing the amino acid sequence shown in SEQ ID NO: 4 or the amino acid sequence encoded by the insertion of plasmid DSM 14633;

(c2) nucleic acid molecules whose sequence has at least 90% homology with the nucleic acid molecules mentioned under (a2) or (b2);

(d2) nucleic acid molecules whose sequence is degenerate in comparison with the sequences of the nucleic acid molecules mentioned under (a2), (b2) or (c2) owing to the genetic code; and

(e2) parts or fragments of the nucleic acid molecules mentioned under a2) to d2) selected from the group consisting of fragments with a length of at least 25, 50, 100 bp, preferably with a length of at least 250 bp, especially preferably with a length of at least 500 bp.

In one such embodiment, the plant generated in the method according to the invention is preferably a plant of the genus Medicago, especially preferably of the species Medicago sativa. Furthermore, the foreign nucleic acid molecule preferably employed in such an embodiment is a nucleic acid molecule which is a nucleic acid molecule as defined above under (a2) to (d2), part of such a molecule which is long enough in order to achieve the desired effect, or a nucleic acid molecule which is complementary to such a nucleic acid molecule or part thereof.

The sequence shown under SEQ ID NO: 3 originates from Medicago sativa and constitutes a partial cDNA sequence which encodes an R1 protein. The coding region shown has 76% homology with the coding region shown in SEQ ID NO: 7, which encodes the potato R1 protein.

In another preferred embodiment of the method according to the invention, the gene which occurs endogenously in the food plant and which encodes an R1 protein is a gene selected from the group consisting of:

(a3) nucleic acid molecules which encompass the coding region of the nucleotide sequence shown under SEQ ID NO: 5 or the coding region of the insertion in plasmid DSM 14635;

(b3) nucleic acid molecules which encode a protein encompassing the amino acid sequence shown in SEQ ID NO: 6 or the amino acid sequence encoded by the insertion of plasmid DSM 14635;

(c3) nucleic acid molecules whose sequence has at least 90% homology with the nucleic acid molecules mentioned under (a3) or (b3);

(d3) nucleic acid molecules whose sequence is degenerate in comparison with the sequences of the nucleic acid molecules mentioned under (a3), (b3) or (c3) owing to the genetic code; and

(e3) parts or fragments of the nucleic acids mentioned under a3) to d3) selected from the group consisting of fragments with a length of at least 25, 50, 100 bp, preferably with a length of at least 250 bp, especially preferably with a length of at least 500 bp.

In one such embodiment, the fodder plant generated in the method according to the invention is preferably a fodder plant of the genus Lolium, especially preferably of the species Lolium perenne. Furthermore, the foreign nucleic acid molecule preferably employed in such an embodiment is a nucleic acid molecule which is a nucleic acid molecule as defined above under (a3) to (d3), part of such a molecule which is long enough in order to achieve the desired effect, or a nucleic acid molecule which is complementary to such a nucleic acid molecule or part thereof.

The sequence shown under SEQ ID NO: 5 originates from Lolium perenne and constitutes a partial cDNA sequence which encodes an R1 protein. The coding region shown has 75% homology with the coding region shown in SEQ ID NO: 7, which encodes the potato R1 protein.

The present invention also relates to the transgenic fodder plants generated by the method according to the invention, which have an increased leaf starch content in comparison with wild-type plants. The leaf starch content of the fodder plants according to the invention is increased by at least 10-50%, preferably by at least 50-100%, in particular by at least 100-500% and very particularly preferably by at least 500-1 000% in comparison with the corresponding wild-type plants.

In a preferred embodiment, the fodder plants according to the invention are furthermore characterized in that the leaf starch synthesized in them has a reduced phosphate content in comparison with leaf starch from corresponding wild-type plants. In this context, the term “reduced phosphate content” refers to the fact that the phosphate content is reduced by at least 25%, especially preferably by at least 50% and very especially preferably by at least 100% in comparison to the phosphate content of the leaf starch from corresponding wild-type plants. The phosphate content of the leaf starch can be determined by methods known to the skilled worker. It is preferably determined as described in the examples section which follows.

The present invention also relates to cells of a fodder plant according to the invention, the cells being transgenic and genetically modified by the introduction of a foreign nucleic acid molecule whose presence or expression leads to a reduced activity of an R1 protein which occurs endogenously in the fodder plant.

In connection with the transgenic fodder plants and plant cells according to the invention, what has already been said above in connection with the method according to the invention also applies to the preferred embodiments.

A multiplicity of techniques is available for introducing the foreign nucleic acid molecule into a plant host cell in accordance with step (a) of the method according to the invention. These techniques encompass the transformation of plant cells with T-DNA using Agrobacterium tumefaciens or Agrobacterium rhizogenes as transformation agent, protoplast fusion, injection, the electroporation of DNA, the introduction of the DNA by means of the biolistic approach, and other possibilities.

The use of agrobacteria-mediated transformation of plant cells has been studied intensively and described sufficiently, for example in EP 120 516; Hoekema, in: The Binary Plant Vector System, Offsetdrukkerij Kanters B. V., Alblasserdam (1985), chapter V, 63-71; Fraley et al., Crit. Rev. Plant Sci. 4, 1-46 and in An et al., EMBO J. 4 (2), (1985), 277-284. As regards the transformation of potato, see, for example, Rocha-Sosa et al., EMBO J. 8 (1), (1989), (23-29).

The transformation of monocotyledonous plants by means of vectors based on Agrobacterium has also been described (Chan et al., Plant Mol. Biol. 22 (1993), 491-506; Hiei et al., Plant J. 6 (1994) 271-282; Deng et al., Science in China 33 (1), (1990), 28-34; Wilmink et al., Plant Cell Reports 11 (1992), 76-80; May et al., Bio/Technology 13 (1995), 486-492; Conner and Domisse, Int. J. Plant Sci. 153 (1992), 550-555; Ritchie et al., Transgenic Res. 2 (1993), 252-265). An alternative system for the transformation of monocotyledonous plants is the transformation by means of the biolistic approach (Wan and Lemaux, Plant Physiol. 104 (1994), 37-48; Vasil et al., Bio/Technology 11 (1993), 1553-1558; Ritala et al., Plant Mol. Biol. 24 (1994), 317-325; Spencer et al., Theor. Appl. Genet. 79 (1990), 625-631), protoplast transformation, the electroporation of partially permeabilized cells, or the introduction of DNA by means of glass fibres. In particular the transformation of maize is described repeatedly in the literature (cf., for example, WO 95/06128, EP 0 513 849, EP 0 465 875, EP 0 292 435; Fromm et al., Biotechnology 8 (1990), 833-839; Gordon-Kamm et al., Plant Cell 2 (1990), 603-618; Koziel et al., Biotechnology 11 (1993), 194-200; Mórocz et al., Theor. Appl. Genet. 80 (1990), 721-726).

The successful transformation of other cereal species has also been described, for example for barley (Wan and Lemaux, above; Ritala et al., above; Krens et al., Nature 296 (1982), 72-74) and for wheat (Nehra et al., Plant J. 5 (2), (1994), 285-297; Altpeter et al., Mol. Breeding 6 (2000), 519-528).

The genetically modified cell may be regenerated into a plant in accordance with step (b) of the method according to the invention by prior-art methods with which the skilled worker is familiar.

If the expression of the foreign nucleic acid molecule in the plant cells is required for achieving the desired effect, i.e. a reduced activity of the R1 protein, any promoter which is active in plant cells is suitable for this purpose. The promoter can be chosen in such a way that expression in the plants is constitutive or only takes place in a particular tissue, at a particular point in time of plant development or at a point in time determined by external factors. With regard to the plant, the promoter can be homologous or heterologous. Useful promoters are, for example, the cauliflower mosaic virus 35S RNA promoter, the maize ubiquitine promoter and the actin promoter for constitutive expression, or a promoter which ensures expression only in photosynthetically active tissues, for example the ST-LS1 promoter (Stockhaus et al., Proc. Natl. Acad. Sci. USA 84 (1987), 7943-7947; Stockhaus et al., EMBO J. 8 (9), (1989), 2445-2451), the Ca/b promoter (see, for example, U.S. Pat. No. 5,656,496; U.S. Pat. No. 5,639,952; Bansal et al., Proc. Natl. Acad. Sci. USA 89 (1992), 3654-3658) and the Rubisco SSU promoter (see, for example, U.S. Pat. No. 5,034,322 or U.S. Pat. No. 4,962,028), and inducible promoters.

Furthermore, the foreign nucleic acid molecule may contain a termination sequence which serves for the correct termination of transcription and for the addition of a poly-A tail to the transcript, which is thought to have a function in stabilizing the transcripts. Such elements are described in the literature (cf., for example, Gielen et al., EMBO J. 8 (1), (1989), 23-29) and can be exchanged as desired.

The generation of further plants in accordance with step (c) of the method according to the invention can be carried out by any suitable method, for example by vegetative propagation (for example via cuttings, tubers or via callus culture and regeneration of intact plants) or by sexual propagation. Sexual propagation preferably takes place under controlled circumstances, i.e. selected plants which have specific characteristics are hybridized with each other and propagated. The present invention also relates to the plants which can be obtained by this type of propagation.

The present invention also encompasses propagation material of the fodder plants according to the invention, comprising the plant cells according to the invention. For the purposes of the present invention, the term “propagation material” encompasses those components of the plant which are suitable for generating progeny via the vegetative or generative route. Examples which are suitable for vegetative propagation are cuttings, callus cultures or rhizomes. Other propagation material encompasses, for example, fruit, seeds, seedlings, protoplasts, cell cultures and the like. The preferred propagation materials is seeds.

Moreover, the present invention also encompasses the use of nucleic acid molecules which encode an R1 protein, or of parts of these, for reducing the activity of an R1 protein in fodder plants, and to the use of such nucleic acid molecules for generating transgenic fodder plants with an increased leaf starch content in comparison with wild-type plants.

The present invention furthermore relates to nucleic acid molecules encoding an R1 protein from plants of the genus Trifolium, selected from the group consisting of

(a) nucleic acid molecules which encode a protein encompassing the amino acid sequence shown in SEQ ID NO: 2;

(b) nucleic acid molecules which encompass the nucleotide sequence, shown in SEQ ID NO: 1, of the coding region;

(c) nucleic acid molecules which encode a protein which encompasses the amino acid sequence encoded by the insertion of plasmid DSM 14707;

(d) nucleic acid molecules which encompass a region, of the insertion of plasmid DSM 14707, which encodes a Trifolium repens R1 protein;

(e) nucleic acid molecules whose sequence has at least 85%, by preference at least 90%, preferably at least 95%, especially preferably at least 97% and very especially preferably at least 99% identity with the sequence of a nucleic acid molecule of (a), (b), (c) and/or (d) and which nucleic acid molecules encode an R1 protein from a plant of the genus Trifolium; and

(f) nucleic acid molecules whose nucleotide sequence deviates from the sequence of a nucleic acid molecule of (e) owing to the degeneracy of the genetic code.

The nucleic acid sequence shown in SEQ ID NO: 1 is a partial cDNA sequence which encompasses part of the coding region for a Trifolium repens R1 protein. A plasmid comprising this cDNA sequence was deposited as DSM 14707. This sequence, or this molecule, now enables the skilled worker to isolate homologous sequences from other Trifolium varieties. This can be done for example with the aid of conventional methods, such as screening cDNA libraries or genomic libraries with suitable hybridization probes. In this manner, it is possible, for example, to identify and isolate nucleic acid molecules which hybridize, and have at least 85% identity, with the sequence shown under SEQ ID NO: 1 and which encode a Trifolium R1 protein.

In principle, the nucleic acid molecules according to the invention can encode an R1 protein from any desired plant of the genus Trifolium; preferably, they encode a Trifolium repens R1 protein.

The present invention furthermore relates to nucleic acid molecules encoding an R1 protein from plants of the genus Medicago, selected from the group consisting of

(a) nucleic acid molecules which encode a protein encompassing the amino acid sequence shown in SEQ ID NO: 4;

(b) nucleic acid molecules which encompass the nucleotide sequence, shown in SEQ ID NO: 3, of the coding region;

(c) nucleic acid molecules which encode a protein which encompasses the amino acid sequence encoded by the insertion of plasmid DSM 14633;

(d) nucleic acid molecules which encompass a region, of the insertion of plasmid DSM 14633, which encodes a Medicago sativa R1 protein;

(e) nucleic acid molecules whose sequence has at least 80%, by preference at least 85%, preferably at least 90%, especially preferably at least 95% and very especially preferably at least 99% identity with the sequence of a nucleic acid molecule of (a), (b), (c) and/or (d) and which nucleic acid molecules encode an R1 protein from a plant of the genus Medicago; and

(f) nucleic acid molecules whose nucleotide sequence deviates from the sequence of a nucleic acid molecule of (e) owing to the degeneracy of the genetic code.

The nucleic acid sequence shown in SEQ ID NO: 3 is a partial cDNA sequence which encompasses part of the coding region for a Medicago sativa R1 protein. A plasmid comprising this cDNA sequence was deposited as DSM 14633. This sequence, or this molecule, now enables the skilled worker to isolate homologous sequences from other Medicago species or varieties. This can be done for example with the aid of conventional methods, such as screening cDNA libraries or genomic libraries with suitable hybridization probes. In this manner, it is possible, for example, to identify and isolate nucleic acid molecules which hybridize, and have at least 80% identity, with the sequence shown under SEQ ID NO: 3 and which encode a Medicago R1 protein.

In principle, the nucleic acid molecules according to the invention can encode an R1 protein from any desired plant of the genus Medicago; preferably, they encode a Medicago sativa R1 protein.

The present invention furthermore relates to nucleic acid molecules encoding an R1 protein from plants of the genus Lolium, selected from the group consisting of

(a) nucleic acid molecules which encode a protein encompassing the amino acid sequence shown in SEQ ID NO: 6;

(b) nucleic acid molecules which encompass the nucleotide sequence, shown in SEQ ID NO: 5, of the coding region;

(c) nucleic acid molecules which encode a protein which encompasses the amino acid sequence encoded by the insertion of plasmid DSM 14635;

(d) nucleic acid molecules which encompass a region, of the insertion of plasmid DSM 14635, which encodes a Lolium perenne R1 protein;

(e) nucleic acid molecules whose sequence has at least 90%, by preference at least 95%, preferably at least 97%, especially preferably at least 99% identity with the sequence of a nucleic acid molecule of (a), (b), (c) and/or (d) and which nucleic acid molecules encode an R1 protein from a plant of the genus Lolium; and

(f) nucleic acid molecules whose nucleotide sequence deviates from the sequence of a nucleic acid molecule of (e) owing to the degeneracy of the genetic code.

The nucleic acid sequence shown in SEQ ID NO: 5 is a partial cDNA sequence which encompasses part of the coding region for a Lolium perenne R1 protein. A plasmid comprising this DNA sequence was deposited as DSM 14635. This sequence, or this molecule, now enables the skilled worker to isolate homologous sequences from other Lolium species or varieties. This can be done for example with the aid of conventional methods, such as screening cDNA libraries or genomic libraries with suitable hybridization probes. In this manner, it is possible, for example, to identify and isolate nucleic acid molecules which hybridize, and have at least 90% identity, with the sequence shown under SEQ ID NO: 5 and which encode a Lolium R1 protein.

In principle, the nucleic acid molecules according to the invention can encode an R1 protein from any desired plant of the genus Lolium; preferably, they encode a Lolium perenne R1 protein.

The nucleic acid molecules according to the invention can take the form of any desired nucleic acid molecules, in particular DNA or RNA molecules, for example cDNA, genomic DNA, mRNA and the like. They may be naturally occurring molecules or else molecules generated by recombinant or chemical synthetic methods. They may take the form of simplexes, which comprise either the coding or the noncoding strand, or duplexes.

The invention furthermore relates to vectors, in particular plasmids, cosmids, viruses, bacteriophages and other vectors conventionally used in genetic engineering which comprise the above-described nucleic acid molecules according to the invention.

In a preferred embodiment, the nucleic acid molecules which are present in the vectors are linked in sense orientation to regulatory elements which ensure expression in prokaryotic or eukaryotic cells. In this context, the term “expression” may refer to transcription or else to transcription and translation.

Expression of the nucleic acid molecules according to the invention in prokaryotic cells, for example in Escherichia coli, makes possible for example a more in-depth characterization of the biological activities of the encoded proteins. In addition, various types of mutations can be introduced into the nucleic acid molecules according to the invention by means of customary molecular-biological techniques (see, for example, Sambrook and Russell, loc. cit.), resulting in the synthesis of the proteins whose biological properties may have been altered. A possibility is the generation of deletion mutants, in which the consecutive deletions from the 5′ or from the 3′ end of the coding DNA sequence give rise to nucleic acid molecules which lead to the synthesis of correspondingly truncated proteins. Another possibility is the introduction of point mutations. The recombinant manipulation in prokaryotic cells can be carried out by methods known to the skilled worker (cf. Sambrook and Russell, loc. cit.).

Regulatory sequences for expression in prokaryotic organisms, for example E. coli, and in eukaryotic organisms have been described widely in the literature, in particular those for expression in yeast, such as, for example, Saccharomyces cerevisiae. An overview of various systems for expressing proteins in various host organisms is found, for example, in Duffaud et al. (Methods in Enzymology 153 (1987), 383-516) and in Bitter et al. (Methods in Enzymology 153 (1987), 516-544).

In a further embodiment, the invention relates to host cells, in particular prokaryotic or eukaryotic cells, which have been transformed with an above-described nucleic acid molecule or a vector, and to cells which are derived from such host cells and which comprise the above-described nucleic acid molecules or vectors. The host cells can be bacterial cells (for example E. coli) or fungal cells (for example yeast, in particular S. cerevisiae), or else plant or animal cells. In this context, the term “transformed” is understood as meaning that the cells according to the invention are genetically modified with a nucleic acid molecule according to the invention in as far as they comprise at least one nucleic acid molecule according to the invention in addition to their natural genome. The nucleic acid molecule according to the invention may be present in the cell in free form, if appropriate in the form of a self-replicating molecule, or may be present as a stable integration into the genome of the host cell. Preferably, the host cells are plant cells.

The present invention furthermore relates to methods for producing an R1 protein in which host cells according to the invention are grown under conditions which permit expression of the protein, and the protein is obtained from the culture, i.e. from the cells and/or the culture medium.

The present invention furthermore relates to a method for producing an R1 protein from plants of the genus Trifolium, Medicago or Lolium, the protein being produced in an in-vitro transcription and translation system using a nucleic acid molecule according to the invention. Such systems are known to the skilled worker.

The invention also relates to proteins which are encoded by the nucleic acid molecules according to the invention or which can be obtained by a method according to the invention.

The present invention furthermore relates to antibodies which specifically recognize a protein according to the invention. These antibodies may be for example monoclonal or polyclonal. They may also be fragments of antibodies which specifically recognize proteins according to the invention. Methods for preparing such antibodies or fragments are known to the skilled worker.

In particular, the present invention also relates to transgenic plant cells which comprise the nucleic acid molecules or vectors according to the invention. Preferably, the cells according to the invention are characterized in that the nucleic acid molecule according to the invention which has been introduced is stably integrated into the genome and under the control of a promoter which is active in plant cells, preferably a promoter which is heterologous with regard to the nucleic acid molecule.

A multiplicity of promoters is available for expressing a nucleic acid molecule according to the invention in plant cells. In principle, any promoter which is functional in the plants chosen for the transformation may be used. The promoter may be homologous or heterologous with regard to the plant species. Suitable examples are the cauliflower mosaic virus 35S promoter (Odell et al., Nature 313 (1985), 810-812), which ensures constitutive expression in all tissues of a plant, and the promoter is construct is described in WO 94/01571. Another example is the promoters of the polyubiquitine genes from maize. However, promoters which are activated only at a point in time which is determined by external influences may also be used (see, for example, WO 93/07279). In this context, promoters which may be of particular interest are promoters of heat shock proteins, which permit simple induction. Furthermore, those promoters which, in a particular tissue of the plant, lead to the expression of downstream sequences may be used (see, for example, Stockhaus et al., EMBO J. 8 (9), (1989), 2445-2451), for example the ST-LS1 promoter, which is only active in photosynthetically active tissue (Stockhaus et al., Proc. Natl. Acad. Sci. USA 84 (1987), 7943-7947). Other promoters which may be mentioned are those which are active in the starch-storing organs of plants to be transformed. These organs are, for example, the maize kernels in the case of maize and the tubers in the case of potatoes. The tuber-specific B33 promoter (Rocha-Sosa et al., EMBO J. 8 (1), (1989), 23-29) is an example which may be used for overexpressing the nucleic acid molecules according to the invention in potatoes. Seed-specific promoters have already been described for different plant species, for example the Vicia faba USP promoter, which ensures seed-specific expression in V. faba and other plants (Fiedler et al., Plant Mol. Biol. 22 (1993), 669-679; Bäumlein et al., Mol Gen. Genet. 225 (1991), 459-467). In maize, promoters which ensure specific expression in the endosperm of the maize kernels are, for example, promoters of the zein genes (Pedersen et al., Cell 29 (1982), 1015-1026; Quattrocchio et al., Plant Mol. Biol. 15 (1990), 81-93).

Thus, the expression of the nucleic acid molecules according to the invention in plant cells is possible.

The present invention thus also relates to a method for generating transgenic plant cells, comprising the introduction of a nucleic acid molecule or vector according to the invention into plant cells. Various plant transformation systems are available to the skilled worker; they have already been mentioned above.

When the nucleic acid molecules according to the invention are expressed in plants, it is possible, in principle, that the protein synthesized may be localized in any desired compartment of the plant cell. To achieve localization in the specific compartment, it may be necessary to link the coding region with DNA sequences which ensure localization in the compartment in question. Such sequences are known (see, for example, Braun, EMBO J. 11 (9), (1992), 3219-3227; Wolter, Proc. Natl. Acad. Sci. USA 85 (1988), 846-850; Sonnewald, Plant J. 1 (1), (1991), 95-106; Rocha-Sosa, EMBO J. 8 (1), (1989), 23-29).

The present invention thus also relates to transgenic plant cells which have been transformed with one or more nucleic acid molecule(s) according to the invention, and to transgenic plant cells which are derived from cells transformed thus. Such cells comprise one or more nucleic acid molecule(s) according to the invention, which is/are preferably linked to regulatory DNA elements which ensure transcription in plant cells, in particular with a promoter. Preferably, the promoter is heterologous with regard to the nucleic acid molecule. Such cells can be distinguished from naturally occurring plant cells by the fact that they comprise at least one nucleic acid molecule according to the invention in addition to any copies which may occur endogenously.

The transgenic plant cells can be regenerated into intact plants by techniques with which the skilled worker is familiar. The plants which can be obtained by regeneration of the transgenic plant cells according to the invention are likewise subject-matter of the present invention. Plants comprising the above-described transgenic plant cells are furthermore subject-matter of the invention. In principle, the transgenic plants may be plants of any desired plant species, i.e. both monocotyledonous and dicotyledonous plants, preferably fodder plants as defined above. The invention likewise relates to propagation material and harvested crops of the plants according to the invention, for example fruits, seeds, aerial parts, for example leaves, stalks and the like.

The nucleic acid molecules according to the invention or parts thereof can be employed in an above-described method according to the invention for generating fodder plants with an increased leaf starch content, preferably for generating plants of the genus Trifolium, Medicago and Lolium.

Moreover, the present invention relates to the use of the nucleic acid molecules according to the invention for identifying similar molecules which likewise encode an R1 protein. This can be done using techniques with which the skilled worker is familiar, for example by hybridization, screening gene libraries, amplification by means of suitable primers in a polymerase chain reaction, and the like.

Deposits

The following plasmids prepared and/or used for the purposes of the present invention were deposited at the Deutsche Sammlung von Mikroorganismen (DSM) in Brunswick, Federal Republic of Germany, which is recognized as international depository, in compliance with the provisions of the Budapest Treaty on the international recognition of the deposit of microorganisms for the purposes of patent procedure.

(Deposit number; Deposit date):

| | | | | |
|---|---|---|---|---|
| Plasmid | IR | 116-156 | (DSM 14707) | (17 Dec. 2001) |
| Plasmid | IR | 102-123 | (DSM 14635) | (16 Nov. 2001) |
| Plasmid | CF | 19-49 | (DSM 14633) | (16 Nov. 2001) |

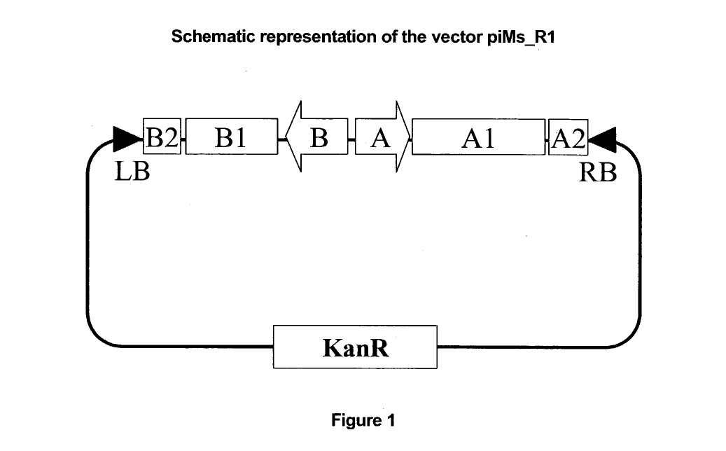

FIG. 1 is a schematic representation of the vector piMs_R1 (Medicago sativa).

| | |
|---|---|
| A: | Cauliflower mosaic virus CaMV 35S promoter, Franck et al., |
| | Cell 21 (1980), 285-294 |
| A1: | Partial R1 cDNA (˜1.9 kbp) from Medicago sativa (antisense |
| | orientation) |
| A2: | Terminator of the Agrobacterium tumefaciens octopine synthase |
| | gene, Gielen et al., EMBO J. 3, (1984) 835-846. |
| B: | Agrobacterium tumefaciens nopaline synthase promoter, Bevan |
| | et al., Nucl. Acids Res. 11 (1983), 369-385 |
| B1: | hph gene, Becker, Nucl. Acids Res. 18 (1990), 203 |
| B2: | Terminator of the nopaline synthase gene, Bevan et al., Nucl. |
| | Acids Res. 11 (1983), 369-385 |
| LB: | T-DNA left border, Gielen et al. (loc. cit.) |
| RB: | T-DNA right border, Gielen et al. (loc. cit.) |
| KanR: | nptIII gene, Trieu-Cout & Courvalin (1983) Gene 23: 331-341 |

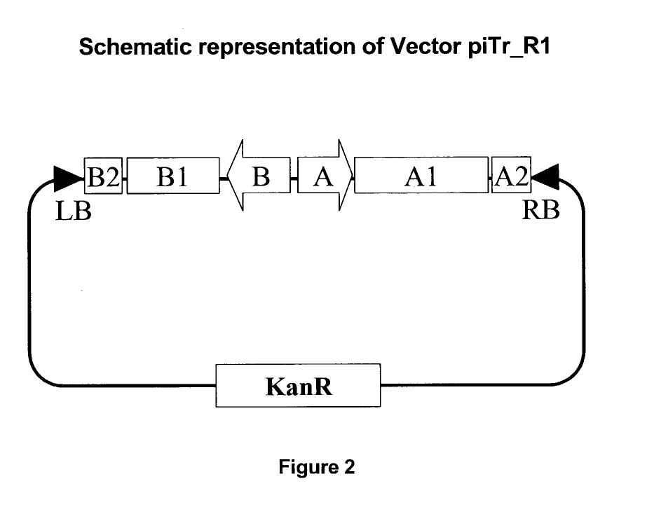

FIG. 2 is a schematic representation of the vector piTr_R1 (Trifolium repens).

| | |
|---|---|
| A: | Arabidopsis thaliana rbc-S promoter, De Almeida et al., Mol. |
| | Gen. Genet. 218 (1989), 78-86 |
| A1: | Partial R1 gene (˜1.1 kbp) from Trifolium repens (antisense |
| | orientation) |
| A2: | nos terminator, Depicker et al., J. Appl. Genet. 1 (1982), |
| | 561-573 |
| B: | CaMV 35S promoter, Franck et al. (loc. cit.) |
| B1: | bar gene, Thompson et al., EMBO J. 6 (1987), 2519-2523 |
| B2: | CaMV 35S terminator, Topfer et al., Nuc. Acids Res. 15 (1987), |
| | 5890 |
| LB: | T-DNA left border, Gielen et al. (loc. cit.) |
| RB: | T-DNA right border, Gielen et al. (loc. cit.) |
| KanR: | nptIII gene, Trieu-Cout & Courvalin, Gene 23 (1983), 331-341 |

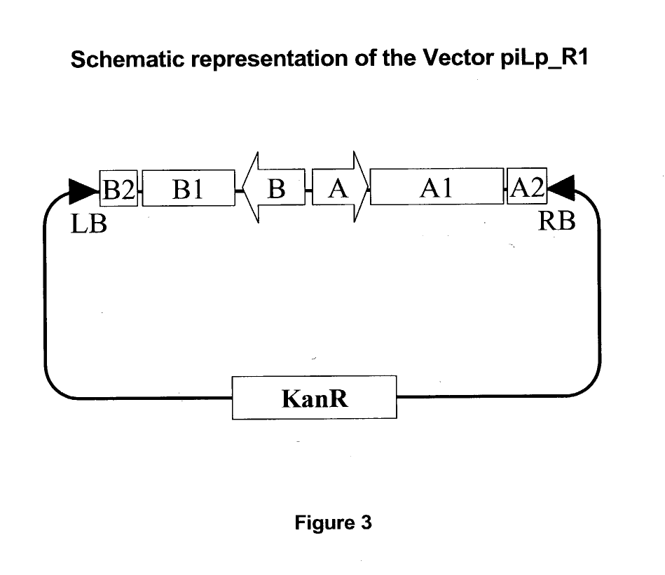

FIG. 3 is a schematic representation of the vector piLp_R1 (Lolium perenne).

| | |
|---|---|
| A: | CaMV 35S promoter, Franck et al. (loc. cit.) |
| A1: | Partial R1 fragment (1.07 kbp) from Lolium perenne (antisense |
| | orientation) |
| A2: | nos terminator, Depicker et al. (loc. cit.) |
| B: | Ubiquitin promoter and first intron from maize, Christensen et |
| | al., Plant Mol. Biol. 18 (1992), 675-689 |
| B1: | npt II gene, Garfinkel et al., Cell 27 (1981), 143-153 |
| B2: | CaMV 35S terminator (loc. cit.) |
| LB: | T-DNA left border (loc. cit.) |
| RB: | T-DNA right border (loc. cit.) |
| KanR: | nptIII gene (loc. cit.) |

The examples which follow illustrate the invention.

### EXAMPLE 1

### Cloning Partial cDNA Sequences of the R1 Protein from Lolium perenne, Trifolium repens and Medicago sativa

The partial cDNA sequences of the R1 protein were cloned from Lolium perenne and Trifolium repens by PCR.

(a) Nucleotide sequences of the primers used for PCR and RT-PCR

| | | |
|---|---|---|
| R1-1: | TACACCTGATATGCCAGATGTTC | (SEQ ID NO: 9) |
| R1-2: | GGCCAYGGCATRCCAGA (Y = C, T; R = A, G) | (SEQ ID NO: 10) |
| Tr R1: | AAGCCCGGGCAAGGAGGGTGAGGATATTGATGACA | (SEQ ID NO: 11) |
| Ms R1-2: | CTACTCACGTTTGATTTGAAGTTGC | (SEQ ID NO: 12) |
| oligodT2: | GAGAGAGTCGAGTTTTTTTTTTTTTTTTTTTTTTTTTT | (SEQ ID NO: 13) |
| Zm_R1-F3: | GAGTGAACTTCAGCAATCAAGTTCTC | (SEQ ID NO: 14) |

ms = Medicago sativum
Tr = Trifolium repens
Zm_R1-F3 = Oligonucleotide whose sequence was deduced from R1 from maize

(b) Isolation of a Genomic Fragment of the DNA of the R1 Gene from Trifolium repens

The following primer combinations were first used for amplifying parts of the genomic sequence of the Trifolium repens R1 gene:

| | | | Size |
|---|---|---|---|
| Primer 1 | Primer 2 | Template | of the amplified fragment |
| R1-1 | R1-2 | DNA from T. repens | 4 kb |

PCR Conditions:

Kit: Platinum Taq Polymerase High Fidelity (Gibco)
approx. 50 ng DNA from T. repens
400 μM primer R1-1 and 400 μM primer R1-2
final concentration 4 mM MgCl₂/200 μM dNTPs

| | | |
|---|---|---|
| 2 min 94° C. denaturation | 30 cycles: | 20 sec 94° C. denaturation |
| | | 30 sec 55° C. hybridization |
| | | 1 min 70° C. elongation |

The plasmid IR 93-123 contains the genomic fragment of the Trifolium repens R1 gene (blunt end cloning of the PCR amplificate by standard methods) in pBluescript SK (Stratagene), linearized using EcoRV.

(c) Cloning a Partial cDNA Fragment Encoding the Trifolium repens R1 Protein

A specific primer (TrR1) was synthesized using the sequence information of IR93-123. To amplify the cDNA fragment by means of RT-PCR, the following two primer combinations were used:

| | | | Size |
|---|---|---|---|
| Primer 1 | Primer 2 | Template | of the amplified fragment |
| Tr R1 | R1-2 | RNA from T. repens | 0.5 kb |
| Tr R1 | ms R1-2 | RNA from T. repens | 1.1 kb |

RT-PCR Conditions:

Kit: One Step RT-PCR Kit from Qiagen
approx. 200 ng of total RNA from T. repens leaves
500 nM primer TrR1 and 500 nM primer Ms R1-2
final concentration 5 mM MgCl₂/200 μM dNTPs

| | | |
|---|---|---|
| 30 min 55° C. RT reaction | 30 cycles: | 15 sec 94° C. denaturation |
| 15 min 95° C. Taq activation | | 30 sec 60° C. hybridization |
| | | 1.5 min 72° C. elongation, |
| | | final 10 min 72° C. |

The plasmid IR 116-156 contains a 1.1 kb cDNA fragment of the Trifolium repens R1 (T/A cloning in pCR2.1 (Invitrogen), following the manufacturer's instructions). The sequence of the cDNA insertion is stated in SEQ ID NO: 1. To generate the plasmid piTr_R1, the approx. 1.07 kb SmaI/XbaI fragment from the plasmid IR 116-156 was cloned into the vector pi-TR downstream of the rbc_S promoter (in antisense orientation), using standard methods.

(d) Cloning a Partial cDNA Fragment Encoding the Lolium perenne R1 Protein

The following primer combination was used for amplifying the cDNA fragment by means of RT-PCR:

| | | | Size of |
|---|---|---|---|
| Primer 1 | Primer 2 | Template | the amplified fragment |
| R1-1 | Oligo dT2 | RNA from L. perenne | 1.2 kb |
| Zm_R1-F3 | Oligo dT2 | RNA from L. perenne | 1.2 kb |

RT-PCR Conditions

Kit: One Step RT-PCR Kit from Qiagen
approx. 200 ng total RNA from L. perenne leaves
500 nM primer R1-1 and 500 nM primer Oligo dT2
final concentration 5 mM MgCl₂/200 μM dNTPs

| | | |
|---|---|---|
| 30 min 55° C. RT reaction | 5 cycles: | 15 sec 94° C. denaturation |
| 15 min 95° C. Taq activation | 25 cycles: | 1.30 min 72° C. elongation |
| | | 15 sec 94° C. denaturation |
| | | 30 sec 60° C. Hybridization |
| | | 1.5 min 72° C. elongation, |
| | | final 10 min 72° C. |

PCR Conditions for the Reamplification:

Kit: Platinum Taq DNA Polymerase High Fidelity (Gibco)

50 ng PCR fragment from RT-PCR

500 nM primer Zm_R1-F3 and 500 nM primer Oligo dT2

Final concentration 5 mM MgSO₄/200 μM dNTPs

2 min 94° C. Taq activation

30 cycles: 15 sec 94° C. denaturation 30 sec 60° C. hybridization 1.5 min 68° C. elongation, final 10 min 72° C.

The plasmid IR 102-123 contains a1.2 kb cDNA fragment of R1 from L. perenne (blunt-end cloning by standard methods into pBluescript SK⁺ (Stratagene) linearized with EcoRV. The sequence of the cDNA insertion is shown in SEQ ID NO: 5. To generate the plasmid piLp_R1, the approx. 1.1 kb XhoI fragment from the plasmid IR102-123 was cloned into the vector pi-Lp downstream of the CaMV 35S promoter (in antisense orientation), using standard methods.

(e) Isolation of a Partial cDNA for the Medicago Sativa R1 Protein

To screen a λZAPII cDNA library of Medicago sativa, 5×10⁵ recombinant phages were plated following the manufacturer's instructions (Stratagene). The phage DNA was transferred to Hybond N filters (Amersham) and immobilized thereon by means of a UV Stratalinker (Stratagene). Prehybridization was carried out for 4 hours at 42° C. (buffer: 5×SSC, 0.5% BSA, 5× Denhardt, 1% SDS, 40 mM phosphate buffer, pH 7.2, 100 mg/l herring sperm DNA, 25% formamide) and subsequently hybridized for 14 hours at the same temperature. The radiolabelled probe (Random Primed DNA Labeling Kit, Boehringer Mannheim, manufacturer's instructions) was the complete cDNA fragment encoding the potato R1 protein (see SEQ ID NO: 7). After hybridization, the filters were washed 3 times for 20 minutes with 3×SSC, 0.5% SDS at 50° C. and autoradiographed for 14 hours. The 6 plaques which showed the highest degree of hybridization were singled out by repeating the screening process three times, and the phages for in-vivo excision were used following the manufacturer's instructions (Stratagene). Plasmid DNA from resulting bacterial colonies was isolated by standard methods (Sambrook and Russell, 2000, loc. cit.) and subjected to DNA sequence analysis. One of the clones (pMs_R1.3) contained an approx. 1.85 kb cDNA fragment with the sequence shown in SEQ ID NO: 3.

(f) Preparation of a Vector for the Antisense Inhibition of the R1 Protein in Medicago sativa

To carry out the antisense inhibition, the complete cDNA fragment was excised from the plasmid pMs_R1.3 by means of the restriction enzymes Asp718 I and SmaI and ligated into the vector pBinAR-Hyg (CaMV 35S/ocs terminator cassette as EcoRI-HindIII fragment in pBIB-Hyg; Becker et al., Nucl. Acids Res. 18 (1990), 203). The resulting plasmid piMs_R1 is shown schematically in FIG. 1.

### EXAMPLE 2

### Plant Transformation

The transformation of Trifolium repens with the vector piTr_R1 was performed by the method described by Larkin et al. (Transgenic Research 5 (1996), 325-335). For the transformation by means of Agrobacterium tumefaciens (comprising plasmid piTr_R1), cotyledons of cultivar Haifa were used. In order to select the transformants, phosphinothricin was added to the B5PB medium in a concentration of 5 mg/l.

The transformation of Medicago sativa with the vector piMs_R1 was performed by the method described by Trinh et al. (Plant Cell Reports 17 (1998), 345-355). For the transformation by means of Agrobacterium tumefaciens (strain GV2260, comprising plasmid piMs_R1), leaf segments of Medicago sativa subspecies falcata (L.) PI.564263 were used.

After the coculture, Agrobacteria were suppressed by adding Ticarpen (500 mg/l) to the SHMab medium. In order to select the transformants, Hygromycin was added at a concentration of 10 mg/l.

The transformation of Lolium repens with the vector piLp_R1 was carried out by the method described by Altpeter et al. (Molecular Breeding 6 (2000), 519-528).

For the transformation, callus material from immature embryos of cultivar L6, which had previously been subcultured for five weeks, was used.

The transformants were selected by culturing the callus material which had been “bombarded” with plasmid piLp_R1 for two weeks on regeneration medium comprising paramomycin at a concentration of 100 mg/l.

### EXAMPLE 3

### Determination of the Starch Content in Leaf Material

(a) Sample Preparation:

Removal of the Soluble Sugars by Extraction with Ethanol:

Approx. 1 g of fresh leaf material from the transgenic plants generated as described in Example 2 was freeze-dried, weighed and subsequently homogenized to a fine powder using a Retsch ball mill. Approx. 50 mg of powdered leaf material (determination in duplicate) were weighed, 1 ml of 80% strength ethanol was added, the mixture was shaken vigorously, and the homogeneous dispersion was incubated for 1 h in a water bath at 80° C. After the dispersion had cooled to approx. 40° C., it was centrifuged for 5 min at 3 000 rpm (Minifuge RF, Heraeus). The supernatant was discarded. The leaf material was treated twice more with in each case 1 ml of 80% strength ethanol and incubated for in each case 20 min in a water bath at 80° C. After cooling and centrifuging (see above), all the supernatants were discarded.

(b) Starch Determination in a Microtitre Plate/Spectramax at 340 nm:

(Stärke Lebensmittelanalytik UV-Test), Boehringer Mannheim, Catalogue No.: 207748 (Amyloglucosidase, starch determination buffer, Glucose-6-phosphate dehydrogenase)

The sugar-free leaf material is treated with 400 microlitres 0.2 N KOH and homogenized by shaking vigorously. The homogenate is incubated for 1 h at 95° C. in a water bath. After cooling, 75 μl 1M acetic acid are added and the reaction mixture is mixed thoroughly. The mixture is centrifuged for 10 min at 4 000 rpm. 25 and 50 μl supernatant are introduced into a microtitre plate containing 50 μl amyloglucosidase (Boehringer Mannheim) and 25 or 50 μl, respectively, of Millipore water and digested for 1 h at 56° C. 196 μl starch determination buffer (Boehringer Mannheim) are introduced into another microtitre plate. To this there are added 4 (to 20) μl of the cooled starch digest. The ratio can be raised to up to 40 μl digest+160 μl starch determination buffer, depending on the glucose concentration.

| | |
|---|---|
| Measurement: | shake, pre-read + |
| | 2 μl glucose-6-phosphate dehydrogenase |
| | (Boehringer Mannheim) |
| | incubation: 30 min at 37° C., measure |

(c) The Starch Content was Calculated as Follows:

Measuring volume (200 μl)×extraction volume (4 750 μl)×amyloglucosidase digest volume (200 μl)×ΔOD/ε×1 000×sample measurement volume (4 μl . . . 40 μl)×sample digest volume (50 μl)×weight (g)×d(1)=concentration (μmol/g DW)

ε=6.3l×mmol⁻¹×cm⁻¹ (molar extinction coefficient of NADH at 340 nm)

DW=dry weight

The concentration in mg glucose/g fresh weight was calculated from the determined weights before and after freeze-drying and the molecular weight of glucose (162.1 g/mol—anhydride).

### EXAMPLE 4

Leaf Starch Extraction

Reagents

| | |
|---|---|
| Extraction buffer pH 7.3: | 50 mM Na-MOPS |
| | MOPS = (3-[N-morpholino]propanesulphonic |
| | acid) |
| | 2 mM EDTA |
| | 0.5 mM beta-mercaptoethanol |
| SDS 2% (Serva) | |
| ethanol 80% | |
| acetone | |

Starch Extraction

Using a Waring blender, the leaves of the plants were comminuted for approx. three minutes at the highest speed, using extraction buffer (8 ml per gram fresh weight). The mixture is subsequently filtered first through a kitchen strainer and then through a 125 μm filter. The solids are again homogenized in the Waring blender using extraction buffer (2 ml per gram fresh weight) and again filtered. After the second extraction, the solids are discarded. The filtrates are combined in a centrifuge bottle and centrifuged for 15 min at 5 500×g. The supernatant is discarded and the pellet is taken up in 2% SDS (8 ml per gram fresh weight). The suspension is filtered through a 30 μm filter with gentle stirring (starch passes through the filter) and then centrifuged for 15 min at 5 500×g. The supernatant is discarded and the pellet is washed three times with water (8 ml per gram fresh weight; resuspended and centrifuged as described above), and all the supernatants are discarded. Again, the pellet is taken up in water (8 ml per gram fresh weight) and the mixture is again filtered through a 30 μm filter, if possible without stirring.

The filtrate is subsequently centrifuged for 15 min at 5 500×g and the pellet is washed twice with 80% ethanol and once with acetone (in each case 0.5 ml per gram fresh weight; resuspended and centrifuged as described above).

After a final wash with water (0.5 ml per gram fresh weight), the pellet (leaf starch) is dried for at least 24 h in a lyophylizer.

Phosphate Determination

Reagents:

| 0.7 N HCl: | 2.9 ml 37% strength HCl/50 ml | |
|---|---|---|
| Buffer: | 9 ml | 1 M imidazole solution pH 7.2 |
| | 225 μl | 1 M MgCl₂ |
| | 60 μl | 0.5 M EDTA |
| | 150 μl | 80 mM NADP |
| | 20.565 ml | Millipore water |
| | 30.0 ml | |

| | |
|---|---|
| Enzyme: | Glucose-6-phosphate dehydrogenase (from Leuconostoc |
| | mesenteroides) |
| | Roche, Catalogue No.: 165875, 1 000 U/1 ml, dilute 1:4 |
| | with buffer |

Sample Preparation:

100 mg leaf starch (weighed accurately) are weighed into a 2 ml Safe-Look Eppendorf tube and treated with 500 μl of 0.7 N HCl. During weighing, the water content of a further 100 mg of leaf starch is determined by means of a temperature-controlled balance.

The mixture is vortexed vigorously; this is followed by acid hydrolysis for 4 hours at 95° C. with shaking. After cooling, the mixture is centrifuged for 20 min at 13 000 rpm. All of the supernatant is transferred into a Spin Module Size 100 (Q.BIOgene) and filtered by briefly centrifuging.

140 μl of hydrolysate are mixed with 1 260 μl of buffer in the Eppendorf tube, and two 700 μl aliquots are transferred into two quartz cuvettes (reference cuvette and sample cuvette). Enzymatic determination of glucose-6-phosphate is started by addition of 6 μl of enzyme to the sample cuvette. The measurement (increase in NADPH) is carried out at 340 nm using a UVIKON apparatus (Kontron).

Calculation:

$$\begin{matrix}\text{Calculation:} & \\ \frac{Measuring volume ( 700 μl ) \times extraction volume ( 500 μ l ) \times delta OD}{ϵ \times sample volume ( 70 μ l ) \times {weight}^{*} ( mg ) \times d ( = 1 )} = \begin{matrix} C6P concentration \\ per dry leaf starch \\ n mol \text{/} mg\end{matrix} & \end{matrix}$$

ε=6.3 l×mmol⁻¹×cm⁻¹ (molar extinction coefficient of NADPH at 340 nm)

d=path length of the cuvette

1. Method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one antisense RNA which brings about reduced expression of endogenous genes which encode R1 proteins.

2. Method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which, via a cosuppression effect, lead to reduced expression of endogenous genes which encode R1 proteins.

3. Method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one ribozyme which specifically cleaves transcripts of endogenous genes which encode R1 proteins.

4. Method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

5. Method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which simultaneously encode at least one antisense RNA and at least one sense RNA, where the antisense RNA and the sense RNA form a RNA duplex which brings about a reduced expression of endogenous genes which encode R1 proteins.

6. Method according to claim 1, where the reduced activity of the R1 protein means an amount of R1 protein which is reduced by at least 50% in comparison with corresponding cells which have not been genetically modified.

7. Method according to claim 1, where the fodder plant is a plant of the genus Trifolium.

8. Method according to claim 1, where the fodder plant is a plant of the genus Medicago.

9. Method according to claim 1, where the fodder plant is a plant of the genus Lolium.

10. Transgenic fodder plant which has an increased leaf starch content and which can be obtained by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one antisense RNA which brings about reduced expression of endogenous genes which encode R1 proteins.

11. Transgenic fodder plant which has an increased leaf starch content and which can be obtained by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which, via a cosuppression effect, lead to reduced expression of endogenous genes which encode R1 proteins.

12. Transgenic fodder plant which has an increased leaf starch content and which can be obtained by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one ribozyme which specifically cleaves transcripts of endogenous genes which encode R1 proteins.

13. Transgenic fodder plant which has an increased leaf starch content and which can be obtained by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

14. Transgenic fodder plant which has an increased leaf starch content and which can be obtained by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which simultaneously encode at least one antisense RNA and at least one sense RNA, where the antisense RNA and the sense RNA form a RNA duplex which brings about a reduced expression of endogenous genes which encode R1 proteins.

15. Transgenic plant cell which is characterized in that it originates from a transgenic fodder plant according to claim 10 and which is genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one antisense RNA which brings about reduced expression of endogenous genes which encode R1 proteins.

16. Transgenic plant cell which is characterized in that it originates from a transgenic fodder plant according to claim 11 and which is genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which, via a cosuppression effect, lead to reduced expression of endogenous genes which encode R1 proteins.

17. Transgenic plant cell which is characterized in that it originates from a transgenic fodder plant according to claim 12 and which is genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one ribozyme which specifically cleaves transcripts of endogenous genes which encode R1 proteins.

18. Transgenic plant cell which is characterized in that it originates from a transgenic fodder plant according to claim 12 and which is genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

19. Transgenic plant cell which is characterized in that it originates from a transgenic fodder plant according to claim 12 and which is genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which simultaneously encode at least one antisense RNA and at least one sense RNA, where the antisense RNA and the sense RNA form a RNA duplex which brings about a reduced expression of endogenous genes which encode R1 proteins.

20. Propagation material of transgenic fodder plants according to claim 10, comprising transgenic plant cells which are characterized in that they are genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one antisense RNA which brings about reduced expression of endogenous genes which encode R1 proteins.

21. Propagation material of transgenic fodder plants according to claim 11, comprising transgenic plant cells which are characterized in that they are genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which, via a cosuppression effect, lead to reduced expression of endogenous genes which encode R1 proteins.

22. Propagation material of transgenic fodder plants according to claim 12, comprising transgenic plant cells which are characterized in that they are genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which encode at least one ribozyme which specifically cleaves transcripts of endogenous genes which encode R1 proteins.

23. Propagation material of transgenic fodder plants according to claim 13, comprising transgenic plant cells which are characterized in that they are genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) nucleic acid molecules which, in the event of in vivo mutagenesis, lead to a mutation or an insertion of a heterologous sequence in endogenous genes which encode R1 proteins, the mutation or insertion leading to reduced expression of genes encoding R1 proteins, or to a reduced synthesis of active R1 proteins.

24. Propagation material of transgenic fodder plants according to claim 14, comprising transgenic plant cells which are characterized in that they are genetically modified with a foreign nucleic acid molecule by a method for generating a transgenic fodder plant with an increased leaf starch content in comparison to corresponding wild-type plants, where
(a) a cell of a fodder plant is genetically modified by introducing a foreign nucleic acid molecule whose presence or expression leads to a reduced activity of an R1 protein which occurs endogenously in the plant cell;
(b) a plant is regenerated from the cell generated in step (a);
(c) if appropriate, further plants are generated starting from the plant generated in step (b); and
(d) the foreign nucleic acid molecule according to step (a) is selected from DNA molecules which simultaneously encode at least one antisense RNA and at least one sense RNA, where the antisense RNA and the sense RNA form a RNA duplex which brings about a reduced expression of endogenous genes which encode R1 proteins.


SEQUENCE LISTING (14 sequences)

SEQ ID NO: 1 - DNA, 1141 bp, Trifolium repens
  CDS (1)..(1053)
aaggagggtgaggatattgatgacaaatcaaccgacctgaaagaagttggttctgttccaactttatctttggtcagaaagcagttcagtggtagatatgctatctcatctgaagaattcactggtgaaatggttggagctaaacctcgtaatatctcttacttgaaaggaaaggtgccttcttgggtaggaattcctacctcggttgccttaccatttggagtttttgaacatgttctctctgataagtcaaatcaggttgtggctgagaaggtcaatattttaaaaaagaagctaactgaaggagacttcagtgccctcaaggagattcgtgaaacagttttagagttgaatgctccacccaagttggtagaggagttgaaaactacaatgaagggttctgggatgccttggccgggtgatgaaggtgaacaacgctggggacaggcatggaaggctataaaaaaagtgtggggctcaaagtggaacgaaagagcgtacttcagcacaagaaaagtaaaactggaccacgattatctttccatgtccgtccttgtacaggaagtgattaatgctgactatgcctttgtcatccacacaaccaatccgacctctggagactcttcagaaatatatacagaggttgtaaagggacttggtgaaacactggttggagcttatcccggccgtgctttgagttttatctgcaaaaaacatgatttgaactctcctcaggtcttgggttatcctagcaaacctatcgggctatttataagacggtcaataatttttcggtctgattccaatggcgaagatctcgaaggttatgctggtgctggtctttatgacagtgtgccaatggatgaagaagagaaagtggtgctagattactcatcagataaactgatgattgatggtagttttcgccagtctatcttgtcaagcattgcccgtgcaggacatgcaatcgaagagttgtatggcactcctcaggacattgaaggtgtcatcaaggatggaaaagtctatgttgtccagaccagaccacaagtgtaggtctctagactcataacagataagctgcacatctcaactacgttcagcaccatactacatgcaacttcaaatcaaacgtgagtag

SEQ ID NO: 2 - protein, 351 aa, Trifolium repens
KEGEDIDDKSTDLKEVGSVPTLSLVRKQFSGRYAISSEEFTGEMVGAKPRNISYLKGKVPSWVGIPTSVALPFGVFEHVLSDKSNQVVAEKVNILKKKLTEGDFSALKEIRETVLELNAPPKLVEELKTTMKGSGMPWPGDEGEQRWGQAWKAIKKVWGSKWNERAYFSTRKVKLDHDYLSMSVLVQEVINADYAFVIHTTNPTSGDSSEIYTEVVKGLGETLVGAYPGRALSFICKKHDLNSPQVLGYPSKPIGLFIRRSIIFRSDSNGEDLEGYAGAGLYDSVPMDEEEKVVLDYSSDKLMIDGSFRQSILSSIARAGHAIEELYGTPQDIEGVIKDGKVYVVQTRPQV

SEQ ID NO: 3 - DNA, 1855 bp, Medicago sativa
  CDS (3)..(1508)
tacttggcgtggaaaattgggctgtggaaatatttactgaagaaattatccgtgctggatctgctgcttctttgtctactcttgtaaatcgactagatccagtactccgaaagactgctaatcttggaagttggcaggttattagcccagttgaaaccgttggatatgttgaggttgtggatgagttgctagctgttcaaaataaaacatatgagcgacctacaattttgatagccaagagcgtgagaggagaggaggaaattccagacggtacagttgctgtcctgacacctgatatgccagatgttctttctcatgtatctgtacgggcaagaaatagcaaggtgtgttttgcgacatgttttgatcccaatatctttgctgacctccaagcaaataaaggaaagctcttgcggttaaagcctacatcagctgaggttgtttatagtgaggtcaaggaaggggagaatattgatgacaaatcaaccgacctgaaagaagttgattctattccatctttatctttggtcaaaaagcagttcagtggtagatatgctatctcttctgaagaattcactggtgaaatggttggagcaaaatcccgtaatatctcgtacttaaaaggaaaggtgccttcttgggttggaattcctacctcggttgccataccatttggagtttttgaacatgttctctctgataagtcaaatcaggctgtggctgcgaagattgatgttttaaaaaagaagttaactgaaggagacttcggtgccctcaaggagattcgtgaaacagttttacagttgaatgcacctcctaagttgatagaggagttgaaaactacgatgaagggttctgggatgccttggccgggggatgaaggtgaaaaacgctggggacaggcatggacggctataaaaaaagtctggggatcaaagtggaatgaaagagcgtacttcagcacaagaaaagtgaagctggaccacgattatctttccatgtcagtccttgtacaagaagtgattaatgctgactatgcttttgtcatccacacaactaacccgacctctggagactcttcagaaatatatactgaggttgtaaagggactgggtgaaacactggttggagcttatcccggccgtgctttgagttttatctgcaaaaaacaagatttgaactctcctcaggtcttgggttatcctagcaaacctattgggctatttataagacggtcaataatttttcgatctgattccaatggtgaagatctcgaaggttacgctggtgctggtctatatgacagtgtgccaatggatgaagaagagaaagtggtgctagattactcgtcagataaactaatgactaatagcagttttcgccagtcaatcttgtcaagcattgccagtgcaggaaatgcaatcgaagagttgtacggcactcctcaggacattgaaggtgttgtcaaggatggaaaaatctatgttgtccagaccagaccacaagtgtaggtctctagtctcattacagctaagccacacatctcaactaaatttgtcaccatattgcacggcaacttcaaatcaaacgtgagtagcttcttgcaaaattgagagctttggtttgtacttttaatgtaactgatatatatgaaaataaagaataatttacgtgtaactatagaataatgtaattatggaaacatgcaaatgtttctattttccccccctcttactatatgtaaaatttgaatcctattcttctcagtcgatgttgtatttttacttgatgtctactgtcaatggttagataataaaatacacagaaatttattaaaaaaaaaaaaaaaaaaaaa

SEQ ID NO: 4 - protein, 502 aa, Medicago sativa
LGVENWAVEIFTEEIIRAGSAASLSTLVNRLDPVLRKTANLGSWQVISPVETVGYVEVVDELLAVQNKTYERPTILIAKSVRGEEEIPDGTVAVLTPDMPDVLSHVSVRARNSKVCFATCFDPNIFADLQANKGKLLRLKPTSAEVVYSEVKEGENIDDKSTDLKEVDSIPSLSLVKKQFSGRYAISSEEFTGEMVGAKSRNISYLKGKVPSWVGIPTSVAIPFGVFEHVLSDKSNQAVAAKIDVLKKKLTEGDFGALKEIRETVLQLNAPPKLIEELKTTMKGSGMPWPGDEGEKRWGQAWTAIKKVWGSKWNERAYFSTRKVKLDHDYLSMSVLVQEVINADYAFVIHTTNPTSGDSSEIYTEVVKGLGETLVGAYPGRALSFICKKQDLNSPQVLGYPSKPIGLFIRRSIIFRSDSNGEDLEGYAGAGLYDSVPMDEEEKVVLDYSSDKLMTNSSFRQSILSSIASAGNAIEELYGTPQDIEGVVKDGKIYVVQTRPQV

SEQ ID NO: 5 - DNA, 1077 bp, Lolium perenne
  CDS (3)..(827)
tcgagaaggtcttgccagatgataccaataaggaagtagcacaaaacatacagatgctgaagggtagacttgatcaagatgattttagtgctcttggagaaatgcggaaaactgttcttgatttaactgctccagctcaactggttacagagctgaaggagaagatgctaagttctggaatgccctggcctggggatgaaagtgaccagcgctgggagcaagcatggatggcaattaaaaaggtctgggcatcaaaatggaacgaaagagcatattttagcacacgcaaggtgaagctcgatcatgactacctttccatggctgttcttgtacacgaaattgtcaacgcagactatgccttcgtcattcatactacgaacccgtcatctggagattcttccgagatatacgctgaagtggtgaaaggacttggagagacacttgtgggagcctatcctggccgagccatgagcttcgtgtgtaagaaagatgaccttgactctcccaaggtactgggttacccaagtaagccaattggtctcttcataaagcgatcaatcatcttccgttcagattctaatggtgaggatctggaaggttatgctggagcaggactgtatgatagtgtccctatggatgaggaagatcaagttgtgctcgactacacggccgacgctctcatcacggactctggattccgaagctcaattctctcaagcattgcacgggctggccatgccattgaggagctctacggttcaccacaggacgttgagggagtagtcaaggatgggaagatctacgtagtccagactagaccacagatgtaatacgtacctataggcagctcaagttgtagagtagtgggatatatgagcctttatggcatgcatagttgtacaacacatcatacatgtatgctcagaataaggtttaaccatatactgtattttctggagcttgtatttgcaccaataaagtgtatgtatcatgaggaatatgaacctactaaaaaattcagaggaaacatttttgagcaataaaaaaaaaaaaaaaaaaaaaaaaaaaaaaaaaaaa

SEQ ID NO: 6 - protein, 275 aa, Lolium perenne
EKVLPDDTNKEVAQNIQMLKGRLDQDDFSALGEMRKTVLDLTAPAQLVTELKEKMLSSGMPWPGDESDQRWEQAWMAIKKVWASKWNERAYFSTRKVKLDHDYLSMAVLVHEIVNADYAFVIHTTNPSSGDSSEIYAEVVKGLGETLVGAYPGRAMSFVCKKDDLDSPKVLGYPSKPIGLFIKRSIIFRSDSNGEDLEGYAGAGLYDSVPMDEEDQVVLDYTADALITDSGFRSSILSSIARAGHAIEELYGSPQDVEGVVKDGKIYVVQTRPQM

SEQ ID NO: 7 - DNA, 4826 bp, Solanum tuberosum
  CDS (104)..(4495)
atcttcatcgaatttctcgacgcttcttcgctaatttcctcgttacttcactagaaatcgacgtttctagctgaacttgagtgaattaagccagtgggaggatatgagtaattccttagggaataacttgctgtaccagggattcctaacctcaacagtgttggaacataaaagtagaatcagtcctccttgtgttggaggcaattctttgtttcaacaacaagtgatctcgaaatcacctttatcaactgagtttcgaggtaacaggttaaaggtgcagaaaaagaaaatacctatgggaaagaaccgtgctttttctagttctcctcatgctgtacttaccactgatacctcttctgagctagcagaaaagttcagtctagaagggaatattgagctacaggttgatgttaggcctcccacttcaggtgatgtgtcctttgtggattttcaagctacaaatggtagtgataaactgtttttgcactggggggcagtaaagttcggaaaagaaacatggtctcttcctaatgatcgtccagatgggaccaaagtgtacaagaacaaagcacttagaactccatttgttaaatctggctctaactccatcctgagactggagatacgggacactgctatcgaagctattgagtttctcatatacgatgaagcctacgataaatggataaagaataatggtggcaattttcgtgtcaaattgtcaagaaaagagatacgaggcccagatgtttcagttcctgaggagcttgtacagatccaatcatatttgaggtgggagaggaagggaaaacagaattacacccctgagaaagagaaggaggaatatgaggctgctcgaactgagctacaggaggaaatagctcgtggtgcttccatacaggacattcgagcaaggctaacaaaaactaatgataaaagtcaaagcaaagaagagcctcttcatgtaacaaagagtgaaatacctgatgaccttgcccaagcacaagcttacattaggtgggagaaagcaggaaagccgaactatcctccagaaaagcaaattgaagaactcgaagaagcaagaagagaattgcaacttgagcttgagaaaggcattacccttgatgagttgcggaaaaagattacaaaaggggagataaaaactaaggcggaaaagcacgtgaaaagaagctcttttgccgttgaaagaatccaaagaaagaagagagactttgggcagcttattaataagtatccttccagtcctgcagtacaagtacaaaaggtcttggaagaaccaccagccttatctaaaattaagctgtatgccaaggagaaggaggagcagattgatgatccgatccttaataaaaagatctttaaggtcgatgatggggagctactggtactggtagcaaagtcctctgggaagacaaaagtacatatagctacagatctgaatcagccaattactcttcactgggcattatccaaaagtcgtggagagtggatggtaccaccttcaagcatattgcctcctggatcaattattttagacaaggctgccgaaacacctttttccgccagttcttctgatggtctaacttctaaggtacaatctttggatatagtaattgaagatggcaattttgtggggatgccatttgttcttttgtctggtgaaaaatggattaagaaccaagggtcggatttctatgttgacttcagtgctgcatccaaattagcactcaaggctgctggggatggcagtggaactgcaaagtctttactggataaaatagcagatatggaaagtgaggctcagaagtcatttatgcaccggtttaatattgctgctgacttgatagaagatgccactagtgctggtgaacttggttttactggaattcttgtatggatgaggttcatggctacaaggcaactgatatggaacaaaaactataacgtaaaaccacgtgaaataagcaaggctcaggacagacttacagacttgttgcagaatgctttcaccagtcaccctcaataccgtgaaattttgcggatgattatgtcaactgttggacgtggaggtgaaggggatgtaggacagcgaattagggatgaaattttggtcatccagaggaaaaatgactgcaagggtggtatgatggaagaatggcatcagaaattgcataataatactagtcctgatgatgttgtgatctgtcaggcattgattgactacatcaagagtgattttgatcttggtgtttattggaaaaccctgaatgagaacggaataacaaaagagcgtcttttgagttatgaccgtgctatccattctgaaccgaattttagaggagatcaaaagaatggtcttttgcgtgatttaggtcactatatgagaacattgaaggctgttcattcaggtgcagatcttgagtctgctattgcaaactgcatgggctacaaaactgagggagaaggctttatggttggagtccagataaatcctgtatcaggcttgccatctggctttcagggcctcctccattttgtcttagaccatgtggaagataaaaatgtggaaactcttcttgagggattgctagaggctcgtgaggagcttaggcccttgcttctcaaaccaaacaaccgtctaaaggatctgctgtttttggacatagcacttgattctacagttagaacagcagtagaaaggggatatgaagaattgaacaacgctaatcctgagaaaatcatgtacttcatctccctcgttcttgaaaatctcgcactctctgtggacgataatgaagatcttgtttattgcttgaagggatggaatcaagctctttcaatgtccaatggtggagacaaccattgggctttatttgcaaaagctgtacttgacagaatccgtcttgcacttgcaagcaaggcagagtggtaccatcacttattgcagccatctgccgaatatctaggatcaatccttggggtggaccaatgggctttgaacatatttactgaagaaattatacgtgctggatcagcagcttcattatcctctcttcttaatagactcgatcccgtgcttcggaaaactgcaaatctaggaagttggcagattatcagtccagttgaagccgttggatatgttgtcgttgtggatgagttgctttcagttcagaatgaaatctacaagaagcccacgatcttagtagcaaactctgttaaaggagaggaggaaattcctgatggtgctgttgccctgataacaccagacatgccagatgttctttcacatgtttctgttcgagctagaaatgggaaggtttgctttgctacatgctttgatcccaatatattggctgacctccaagcaaaggaaggaaggattttgctcttaaagcctacaccttcagacataatctatagtgaggtgaatgagattgagctccaaagttcaagtaacttggtagaagctgaaacttcagcaacacttagattggtgaaaaagcaatttggtggttgttacgcaatatcagcagatgaattcacaagtgaaatggttggagctaaatcacgtaatattgcatatctgaaaggaaaagtgccttcctcggtgggaattcctacgtcagtagctcttccatttggagtctttgagaaagtactttcagacgacataaatcagggagtggcaaaagagttgcaaattctgacgaaaaaactatctgaaggagacttcagcgctcttggtgaaattcgcacaacgattttagatctttcagcaccagctcaattggtcaaagagctgaaggaaaagatgcagggttctggcatgccttggcctggtgatgaaggtccaaagcggtgggaacaagcatggatggccataaaaaaggtgtgggcttcaaaatggaatgagagagcatacttcagcacaaggaaggtgaaactggatcatgactatctgtgcatggctgtccttgttcaagaaataataaatgctgattatgcatttgtcattcacacaaccaacccatcttccggagacgactcagaaatatatgccgaggtggtcaggggccttggggaaacacttgttggagcttacccaggacgtgctttgagttttatctgcaagaaaaaggatctcaactctcctcaagtgttaggttacccaagcaaaccgatcggccttttcataaaaagatctatcatcttccgatctgattccaatggggaagatttggaaggttatgccggtgctggcctctacgacagtgtaccaatggatgaggaggaaaaagttgtaattgattactcttccgacccattgataactgatggtaacttccgccagacaatcctgtccaacattgctcgtgctggacatgctatcgaggagctatatggctctcctcaagacatcgagggtgtagtgagggatggaaagatttatgtcgttcagacaagacctcagatgtgatcatattctcgttgtatgttgttcagagaagaccatagatgtgatcatattctcatggtatcagatctgtgaccacttacctcccatgaagttgcctgtatgattatacgtgatccaaagccatcacatcatgttcaccttcagctattggaggagaagtgagaagtaggaattgcaatatgaggaataataagaaaaactttgtagaagttaaattagctgggtatgatatagggagaaatgtgtaaacattgtactatatatagtatacacacgcattatgtatttgcattatgcactgaataatatcgcagcatcaaagaagaaa

SEQ ID NO: 8 - protein, 1464 aa, Solanum tuberosum
MSNSLGNNLLYQGFLTSTVLEHKSRISPPCVGGNSLFQQQVISKSPLSTEFRGNRLKVQKKKIPMGKNRAFSSSPHAVLTTDTSSELAEKFSLEGNIELQVDVRPPTSGDVSFVDFQATNGSDKLFLHWGAVKFGKETWSLPNDRPDGTKVYKNKALRTPFVKSGSNSILRLEIRDTAIEAIEFLIYDEAYDKWIKNNGGNFRVKLSRKEIRGPDVSVPEELVQIQSYLRWERKGKQNYTPEKEKEEYEAARTELQEEIARGASIQDIRARLTKTNDKSQSKEEPLHVTKSEIPDDLAQAQAYIRWEKAGKPNYPPEKQIEELEEARRELQLELEKGITLDELRKKITKGEIKTKAEKHVKRSSFAVERIQRKKRDFGQLINKYPSSPAVQVQKVLEEPPALSKIKLYAKEKEEQIDDPILNKKIFKVDDGELLVLVAKSSGKTKVHIATDLNQPITLHWALSKSRGEWMVPPSSILPPGSIILDKAAETPFSASSSDGLTSKVQSLDIVIEDGNFVGMPFVLLSGEKWIKNQGSDFYVDFSAASKLALKAAGDGSGTAKSLLDKIADMESEAQKSFMHRFNIAADLIEDATSAGELGFTGILVWMRFMATRQLIWNKNYNVKPREISKAQDRLTDLLQNAFTSHPQYREILRMIMSTVGRGGEGDVGQRIRDEILVIQRKNDCKGGMMEEWHQKLHNNTSPDDVVICQALIDYIKSDFDLGVYWKTLNENGITKERLLSYDRAIHSEPNFRGDQKNGLLRDLGHYMRTLKAVHSGADLESAIANCMGYKTEGEGFMVGVQINPVSGLPSGFQGLLHFVLDHVEDKNVETLLEGLLEAREELRPLLLKPNNRLKDLLFLDIALDSTVRTAVERGYEELNNANPEKIMYFISLVLENLALSVDDNEDLVYCLKGWNQALSMSNGGDNHWALFAKAVLDRIRLALASKAEWYHHLLQPSAEYLGSILGVDQWALNIFTEEIIRAGSAASLSSLLNRLDPVLRKTANLGSWQIISPVEAVGYVVVVDELLSVQNEIYKKPTILVANSVKGEEEIPDGAVALITPDMPDVLSHVSVRARNGKVCFATCFDPNILADLQAKEGRILLLKPTPSDIIYSEVNEIELQSSSNLVEAETSATLRLVKKQFGGCYAISADEFTSEMVGAKSRNIAYLKGKVPSSVGIPTSVALPFGVFEKVLSDDINQGVAKELQILTKKLSEGDFSALGEIRTTILDLSAPAQLVKELKEKMQGSGMPWPGDEGPKRWEQAWMAIKKVWASKWNERAYFSTRKVKLDHDYLCMAVLVQEIINADYAFVIHTTNPSSGDDSEIYAEVVRGLGETLVGAYPGRALSFICKKKDLNSPQVLGYPSKPIGLFIKRSIIFRSDSNGEDLEGYAGAGLYDSVPMDEEEKVVIDYSSDPLITDGNFRQTILSNIARAGHAIEELYGSPQDIEGVVRDGKIYVVQTRPQM

SEQ ID NO: 9 - DNA, 23 bp, Artificial sequence
  Oligonucleotide
tacacctgatatgccagatgttc

SEQ ID NO: 10 - DNA, 17 bp, Artificial sequence
  Oligonucleotide
ggccayggcatrccaga

SEQ ID NO: 11 - DNA, 35 bp, Artificial sequence
  Oligonucleotide
aagcccgggcaaggagggtgaggatattgatgaca

SEQ ID NO: 12 - DNA, 25 bp, Artificial sequence
  Oligonucleotide
ctactcacgtttgatttgaagttgc

SEQ ID NO: 13 - DNA, 38 bp, Artificial sequence
  Oligonucleotide
gagagactcgagtttttttttttttttttttttttttt

SEQ ID NO: 14 - DNA, 26 bp, Artificial sequence
  Oligonucleotide
gagtgaacttcagcaatcaagttctc